(19) United States
(12) Patent Application Publication
(10) Pub. No.: US 2026/0334884 A1
(43) Pub. Date: Oct. 29, 2026

(54) OXYGEN SATURATION MEASUREMENT

(71) Applicant: ODI MEDICAL AS, Oslo (NO)

(72) Inventor: Luigino CAPONE, Oslo (NO)

(21) Appl. No.: 19/117,923

(22) PCT Filed: Oct. 3, 2023
(86) PCT No.: PCT/EP2023/077361

§ 371 Date: Apr. 2, 2025

(30) Foreign Application Priority Data
Oct. 3, 2022 (GB) 2214523.9

(51) Int. Cl. A61B 5/00; A61B 5/1455; A61B 5/1495

(52) U.S. Cl. CPC A61B 5/7267; A61B 5/14551; A61B 5/1495

(57) ABSTRACT

A computer-implemented method of processing spectral data determines an estimated value of an oxygen saturation parameter which reduces the effect of melanin in its estimation. The method comprises receiving a spectrum (101) obtained by a spectroscopic method performed on a body surface of a subject (150), inputting the spectrum to a trained machine learning model (102), and generating the estimate of the oxygen saturation parameter using the trained machine learning model (102). The machine learning model (102) is trained by synthetic training data comprising a plurality of pairs of spectra. The training data is used to train the machine learning model (102) to generate a modified version of the spectrum (103) corresponding to a melanin parameter with a predetermined value.

This invention relates to reducing the effect of melanin on estimates of oxygen saturation.

Oxygen saturation is a measure of the fraction of haemoglobin in the blood that is bound to oxygen and it is of vital importance for patient health that these measurements remain above approximately 90%. Pulse oximetry is a widespread and common technique for measuring oxygen saturation and can be used both in clinical settings and in the home. A pulse oximeter typically clips onto the patients finger and transmits visible and near-infrared (NIR) light, through the finger, to be received by a photodetector on the other side. The oxygen saturation measurement is dependent on light absorption which can be determined using the data obtained by the photodetector.

Oxygen saturation measurements should be quick to obtain and accurate so that immediate help can be given to at-risk patients. For instance, oxygen saturation measurements can be used to evaluate whether someone needs help breathing in an emergency situation and may be used to help decide whether a patient should be intubated, for example, during a severe acute COVID-19 infection. In such situations, every minute of delay to starting treatment puts the patient at increased risk.

The applicant has appreciated, however, that there exists a significant problem with the impact of melanin on these oxygen saturation readings. It has been found that pulse oximeters often report oxygen saturation values as higher than they actually are for patients with a higher melanin concentration in their skin. This has serious consequences for clinical outcomes for patients with darker skin tones.

Currently, therefore, it is clear that there are inequitable health outcomes for patients with different skin tones. An alternative solution is needed that provides more reliable and accurate readings than the pulse oximeters which are presently being used in hospitals and homes.

It is known to measure blood oxygen levels through the use of light on a patient's skin, e.g. using diffuse reflectance spectroscopy (DRS). Those skilled in the art will appreciate that DRS provides information regarding the oxygenation of the patient's blood. The patient's skin is illuminated with relatively broadband light (i.e. visible and NIR light) using optical fibre probes, and the light reflected by superficial capillaries in the patient's skin is used to determine the haemoglobin concentration and blood oxygenation.

Melanin, produced by melanocytes cells at the lower part of the epidermal layer, is a pigment responsible for the skin tone and is a strong absorber in the ultraviolet (UV)-visible-NIR range, with an extreme absorbing efficiency at UV and no spectral features at specific wavelengths allowing a pass band filtering approach. In the context of inspecting dermal composition using light and in particular reflectance spectroscopy techniques, the presence of melanin, especially if in large concentrations, affects the recorded spectra acting like a generalized damping factor over all the wavelengths, flattening also parts of the wavelengths where other features (e.g. oxy/deoxy-haemoglobin) are to be found and used for parameter estimation.

Although the distribution/size of melanocytes also plays a role, a first-order approximation suggests that the skin tone, and thus melanin concentration in the epidermis, introduces hard-to-control biases in the estimations of micro-circulation that has more impact when a patient's skin tone is darker.

The present invention aims to address this problem.

When viewed from a first aspect, the invention provides a computer-implemented method of processing spectral data to determine an estimated value of an oxygen saturation parameter, the method comprising:
i) receiving a spectrum obtained by a spectroscopic method performed on a body surface of a subject;
ii) inputting said spectrum to a trained machine learning model, the model having been trained by a method comprising:
   generating or receiving synthetic training data comprising a plurality of pairs of spectra, each pair comprising:
      a first training spectrum determined by a first set of parameters, wherein the first set of parameters are deterministic of the first training spectrum, the first set of parameters comprising a melanin parameter;
      a second training spectrum determined by a second set of parameters, wherein the second set of parameters are deterministic of the second training spectrum; wherein the second set of parameters are the first set of parameters with the melanin parameter set to a predetermined value; and
   using the training data to train the machine learning model to generate a modified version of said spectrum corresponding to said melanin parameter having said predetermined value; and
iii) generating said estimate of the oxygen saturation parameter using said trained machine learning model.

The invention extends to software (and to a non-transitory computer-readable storage medium bearing the same) comprising instructions that, when executed by a computer processing system, cause the computer processing system to: perform any of the methods disclosed herein and/or implement trained machine learning model(s) as disclosed herein.

The invention also extends to a processing system configured to perform any of the methods disclosed herein. Steps disclosed herein may be carried out by hardware (e.g. ASICs or FPGAs or other circuitry) or by software or by a combination of hardware and software. The processing system may comprise one or more processors and a memory storing software for execution by the one or more processors.

Thus, it will be appreciated that, embodiments of the invention provide an improved approach to estimating an oxygen saturation parameter using spectra obtained from spectroscopic methods performed on a body surface of a subject.

The trained machine learning model is trained to help reduce the effect of varying melanin levels on spectra obtained using such spectroscopic methods. This approach allows spectra obtained from spectroscopic methods performed on the body surface of the subject, to be transformed into a standardised, melanin-controlled form. This may help to generate more reliable estimated values of an oxygen saturation parameter (e.g. SmVO₂) by preventing the accuracy of oxygen readings being negatively affected by variations in melanin levels, for example, due to a subject's skin tone.

The applicant has appreciated that such an approach to training a machine learning model is novel and inventive in its own right.

Therefore, when viewed from a second aspect, the present invention provides a computer-implemented method of training a machine learning model, the method comprising:
generating or receiving synthetic training data comprising a plurality of pairs of spectra, each pair comprising:
   a first training spectrum determined by a first set of parameters, wherein the first set of parameters are deterministic of the first training spectrum, the first set of parameters comprising a melanin parameter; and
   a second training spectrum determined by a second set of parameters, wherein the second set of parameters are deterministic of the second training spectrum; wherein the second set of parameters are the first set of parameters with the melanin parameter set to a predetermined value; and
using the training data to train the machine learning model to generate:
   an estimate of a value of an oxygen saturation parameter from a spectrum obtained by a spectroscopic method performed on a body surface of a subject; or
   data suitable for determining said estimate.

When viewed from a third aspect, the invention provides a computer-implemented trained machine learning model, the model having been trained by a method comprising:
generating or receiving synthetic training data comprising a plurality of pairs of spectra, each pair comprising:
   a first training spectrum determined by a first set of parameters, wherein the first set of parameters are deterministic of the first training spectrum, the first set of parameters comprising a melanin parameter; and
   a second training spectrum determined by a second set of parameters, wherein the second set of parameters are deterministic of the second training spectrum; wherein the second set of parameters are the first set of parameters with the melanin parameter set to a predetermined value; and
using the training data to train the model to generate:
   an estimate of a value of an oxygen saturation parameter from a spectrum obtained by a spectroscopic method performed on a body surface of a subject; or
   data suitable for determining said estimate.

The machine learning model is trained on a plurality of pairs of spectra. The first and second training spectra are each generated from a set of parameters. The parameters of the first set of parameters are the same (have the same values as) the second set of parameters except for the melanin parameter which can be different (i.e. the second set of parameters are the first set of parameters with the melanin parameter set to a predetermined value). The first training spectrum, therefore, may have an unknown (e.g. randomised) melanin parameter. The second training spectrum has a melanin parameter which is fixed to a predetermined value. Having the ability to generate two different spectra with just a difference in the melanin parameter allows machine learning to be used to build an algorithm that can reduce or remove the effect of melanin from a given input spectrum.

The term ‘subject’ as used herein includes any human or non-human animal subject, including any human or non-human mammal, bird, fish, reptile, amphibian, etc. However, in preferred embodiments the subject is a human.

The term ‘spectroscopic method’ as used herein includes any method that measures the absorption, reflection, transmission and/or emission of light by matter, e.g. diffuse reflectance spectroscopy or pulse oximetry.

The spectral data could be obtained from any suitable source and will be of a type that is obtained from carrying out a spectroscopic method. However, in a set of embodiments, the method comprises the step of performing the spectroscopic method on a body surface of a subject.

The trained machine learning model may receive as input a spectrum obtained from performing such a spectroscopic method on a subject (e.g. a human patient). The spectrum may be a reflectance spectrum obtained by diffuse reflectance spectroscopy performed on a body surface of a subject. The body surface of the subject would typically be a skin surface of the subject. The trained machine learning model may output an output spectrum, e.g. which represents the corresponding input spectrum having a predetermined melanin parameter.

A ‘deterministic’ software or mathematical model having an output which is determined wholly by its input, may operate in a direct mode or an inverse mode.

The deterministic software or mathematical model may generate synthetic spectra for a given set of parameters in the direct mode. In a set of embodiments, the deterministic software or mathematical model generates, in a direct mode, the first training spectrum from the first set of parameters and the second training spectrum from the second set of parameters.

In an example set of embodiments, the deterministic software or mathematical model is configured to generate a reflectance (spectroscopy) spectrum, as output, from a set of input parameters. In some such embodiments, the reflectance spectrum is a Diffuse Reflectance Spectroscopy (DRS) spectrum.

Advantageously Diffuse Reflectance Spectroscopy is non-invasive and thus a safer, procedure compared to arterial blood gas measurements which involve using needles or cannulas to analyse the blood directly. At present, arterial blood gas measurements are the only tests for oxygen saturation which are unaffected by melanin levels in the skin. However, these tests take much more time to perform than DRS or pulse oximetry which can provide almost immediate oxygen saturation measurements (e.g. on the order of seconds). The applicant has recognised that by removing the effect of melanin on DRS measurements, a much faster, non-invasive, melanin-standardised oxygen saturation estimation technique can be provided. This is crucially important for medical situations where delays to treatment can negatively affect patient outcomes. Furthermore, minimal expert training is required to be able to perform DRS and pulse oximetry compared to arterial blood gas measurements, making embodiments of the invention more accessible and less expensive than arterial blood gas measurements.

Therefore, in some embodiments, the deterministic software or mathematical model is a DRS model, the DRS model being arranged, in a direct mode, to receive as input a set of parameters and to generate as output a synthetic DRS spectrum.

Such models are known in the art and one example is discussed in Farrell, T J et al. “A diffusion theory model of spatially resolved, steady-state diffuse reflectance for the noninvasive determination of tissue optical properties in vivo.” Medical physics vol. 19,4 (1992): 879-88. doi:10.1118/1.596777. Examples of ancillary parts of the model used in embodiments of the present invention, including how melanin may be modelled, can be found here: “Jacques, Steven L. “Optical properties of biological tissues: a review.” Physics in medicine and biology vol. 58,11(2013 ):

R37-61. doi:10.1088/0031-9155/58/11/R37”.

As mentioned above, the deterministic software or mathematical model may also operate in an inverse mode, receiving spectra as input, and outputting an estimated value of one or more parameters (e.g. an oxygen saturation parameter). In embodiments where the deterministic software or mathematical model is a DRS model, the DRS model is arranged, in an inverse mode, to receive as input a DRS spectrum and to generate as output one or more parameters of the set of parameters (e.g. an oxygen saturation parameter).

Therefore, in a set of embodiments, the trained machine learning model is trained to generate a filtered spectrum, wherein the melanin parameter is set to said predetermined value. The filtered spectrum may subsequently be passed to another model or algorithm which may be used to generate an estimate of the oxygen saturation parameter. In a set of embodiments, generating an estimate of oxygen saturation parameter is performed by a deterministic software or mathematical model operating in an inverse mode, the deterministic software or mathematical model being arranged to operate in the inverse mode or in a direct mode.

The oxygen saturation parameter value estimated using embodiments of the invention may be representative of a measure of oxygen saturation-e.g. an SmVO₂ estimate. Therefore, the trained machine learning model may be used in conjunction with a or the deterministic software or mathematical model to obtain an estimate of oxygen saturation (e.g. of a human patient). The oxygen saturation estimate may be determined using non-linear regression for example.

In a set of embodiments, the melanin parameter represents a measure of a melanin concentration in the body surface of the subject (e.g. in the skin). The predetermined value of the melanin parameter represents a hypothetical melanin concentration. The predetermined value (hypothetical melanin concentration) may have a value of between 1% and 5%—e.g. between 2% and 4%—e.g. approximately 3%. The applicant has recognised that it is desirable for this value to be relatively low in order to substantially remove the effect of melanin but that is not necessary to reduce it to zero. A value in the ranges given above allows a comparison with the lowest typical values of naturally occurring melanin.

Advantageously, the predetermined value of the melanin parameter is selected to minimise the effect of the melanin on the spectra. The predetermined value may be a value for which a double peak in a reflectance spectrum is most easily discernible. The predetermined value may be a value which allows an oxygen saturation parameter to be obtained that is substantially standardised for all skin tones, allowing more reliable estimates of oxygen saturation values.

In accordance with the invention, the machine learning model is trained to standardise the effect of melanin on spectral data, which herein may be referred to as ‘demelanising’ the spectra.

In a set of embodiments, the training method is executed offline. Offline machine learning may be faster, more easily controlled, lower cost and may require less computational power than online machine learning.

The trained machine learning model may be trained using a combination of training data and validation data. The training data comprises a plurality of pairs of spectra, each pair comprising a first training spectrum and a second training spectrum which are the same except for the influence of the melanin parameter-which may be the only parameter that differs between the two sets of parameters used to generate the spectra. Similarly, the validation data may comprise a plurality of pairs of spectra, each pair comprising a first validation spectrum and a second validation spectrum.

In a set of embodiments, the machine learning model is trained via unsupervised learning. By using unsupervised learning, the training data and/or validation data do not need to be tagged or labelled. It will be appreciated that this can help to simplify the preparation of training data and/or validation data.

The machine learning model may comprise an unsupervised neural network. In a set of embodiments, the unsupervised neural network is an autoencoder, e.g. comprising two mirrored artificial neural network architectures. The autoencoder may be of either a generative (e.g. variational or adversarial) or non-generative type (e. g lower dimensionality types such as basic, convolutional or LSTM-based;

regularisation types such as sparse and contractive; noise tolerance types such as denoising and robust) and may encompass specialised variants. An autoencoder typically comprises an encoder and a decoder. The encoder may transform an input spectrum into a compressed version and the decoder may provide an output spectrum from the compressed version. The first training spectrum and second training spectrum may act as an input and a target, respectively, for the autoencoder.

It is known in other technical fields to use autoencoders for the purpose of building ‘denoisers’ for images. However, typically prior art techniques involve adding artificial noise to images and then presenting the original image as a target to train the autoencoder to remove that specific kind of statistical error.

This differs to the approach as claimed herein, which instead considers and removes a melanin signal that is superimposed to the reflectance spectrum which would be obtained if there was a fixed concentration of melanin present within the body surface of the relevant subject. This approach is not known in the art per se. In an example set of embodiments, the machine learning model is trained by a method further comprising selecting each of the first set of parameters and the second set of parameters from a space (e.g. grid) of admissible values. Equally, the parameters may be selected using random sampling of values.

The space of admissible values may comprise, for each parameter of the set of parameters, a permitted range of values for each parameter of the set of parameters. Each permitted range may be represented in the grid by a minimum value and a maximum value for each parameter. The space of admissible values may further comprise a permitted resolution for each parameter.

The first set of parameters may contain N parameters which may be selected (randomly or sequentially), e.g. from admissible values. The second set of parameters may also contain N parameters, wherein N-1 of the parameters are identical to the first set of parameters but the melanin parameter (the Nth parameter) is set to a predetermined value.

In a set of example embodiments, each set of parameters may comprise parameters representative of one or more of the group comprising: a scaling factor; calibrating light scattering effects and physiological properties such as the relative amount of blood (blood volume fraction, BVF), oxygen saturation and melanin.

The training data may be prepared by a random or exhaustive search (e.g. a sweep) exploring the space of permitted values of the N parameters, based on the granularity associated for each parameter. In a set of embodiments, each parameter has a granularity of between 10 and 100 levels. For example, a granularity of 50 levels for each parameter (e.g. 2% steps for oxygen saturation) will require only about 15 billion synthetic spectra to be generated.

In a set of embodiments, at least some, preferably all of the training, validation, testing and input spectra are input as square images (e.g. comprising 1024 or 2048 points (e.g. pixels)). This allows the spectrum to be easily transformed into a two-dimensional matrix (e.g. a 32×32 or a 64×64 matrix). This may help to speed up the training process.

It will be appreciated that melanin parameter values may not be completely uniform over the body surface of the subject. Therefore, when performing spectroscopic methods on a body surface, any variation in melanin caused by a non-uniform spatial distribution of melanin locally on said body surface can cause very different oxygen saturation parameters to be estimated. Using embodiments of the invention, removing the influence of melanin, helps to remove the effect of these local variations of melanin on oxygen saturation estimates, helping stabilize the oxygen saturation estimates for a given subject, as well as between different subjects.

Features of any aspect or embodiment described herein may, wherever appropriate, be applied to any other aspect or embodiment described herein.

Where reference is made to different embodiments or sets of embodiments, it should be understood that these are not necessarily distinct but may overlap.

An embodiment of the invention will now be described, by way of example only, with reference to the accompanying drawings, in which:

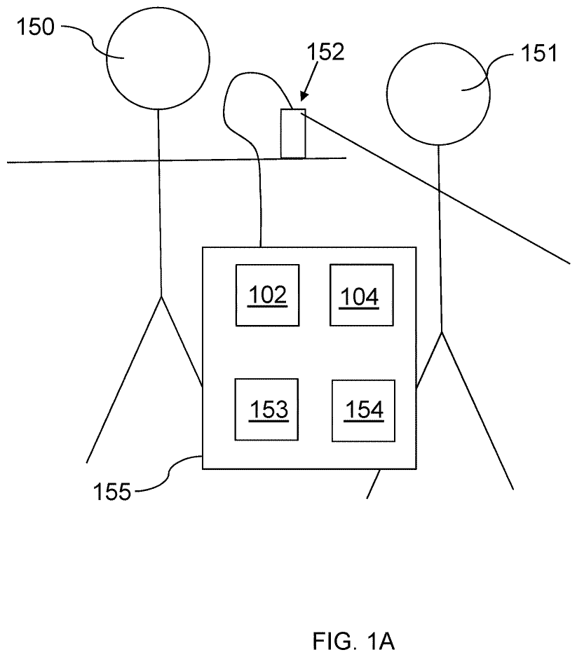

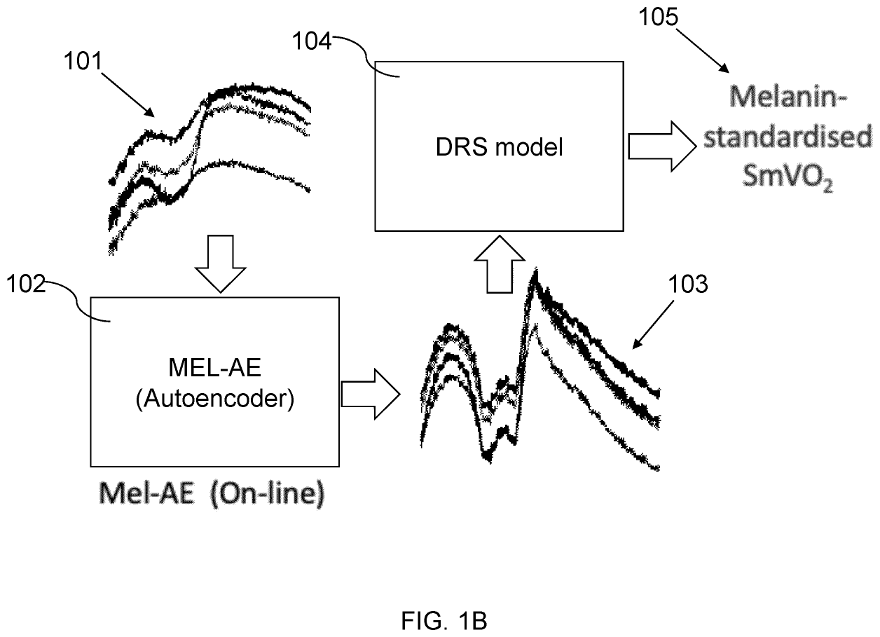

FIG. 1A is a schematic drawing illustrating how an oxygen saturation estimate can be obtained from a subject in accordance with embodiments of the invention;

FIG. 1B is a flow diagram illustrating how DRS spectra can be processed using a trained machine learning model and a deterministic software or mathematical model to give an oxygen saturation parameter estimate, in accordance with embodiments of the invention;

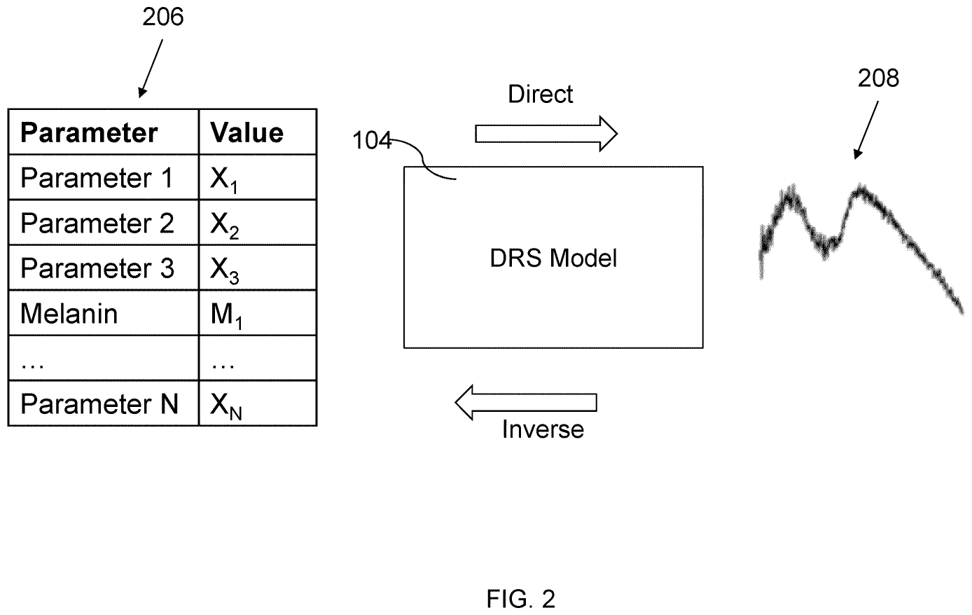

FIG. 2 is a diagram of a DRS model suitable for receiving the output of the trained machine learning model;

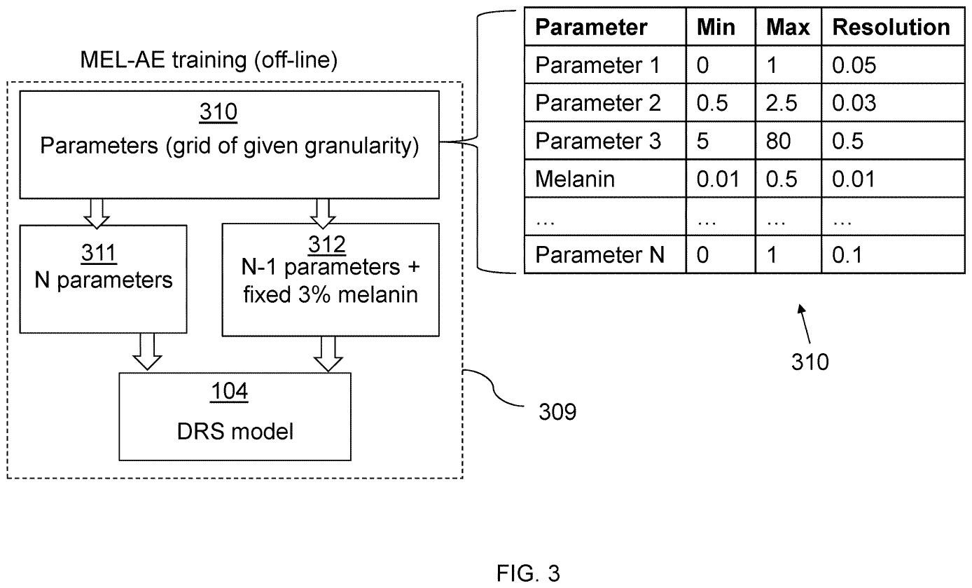

FIG. 3 illustrates how a machine learning model in accordance with embodiments of the invention is trained;

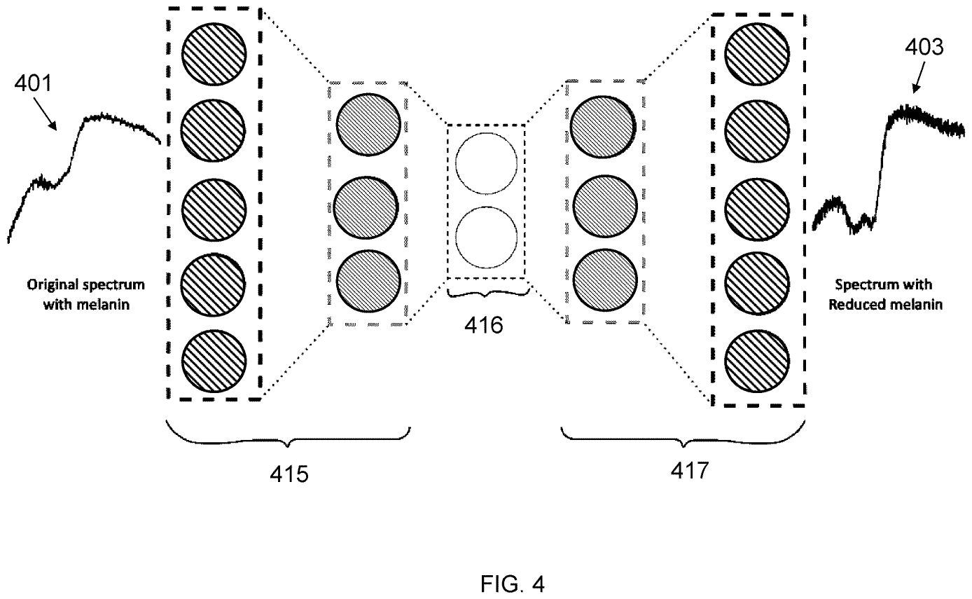

FIG. 4 is a diagram of the trained machine learning model architecture with exemplary input and output spectra;

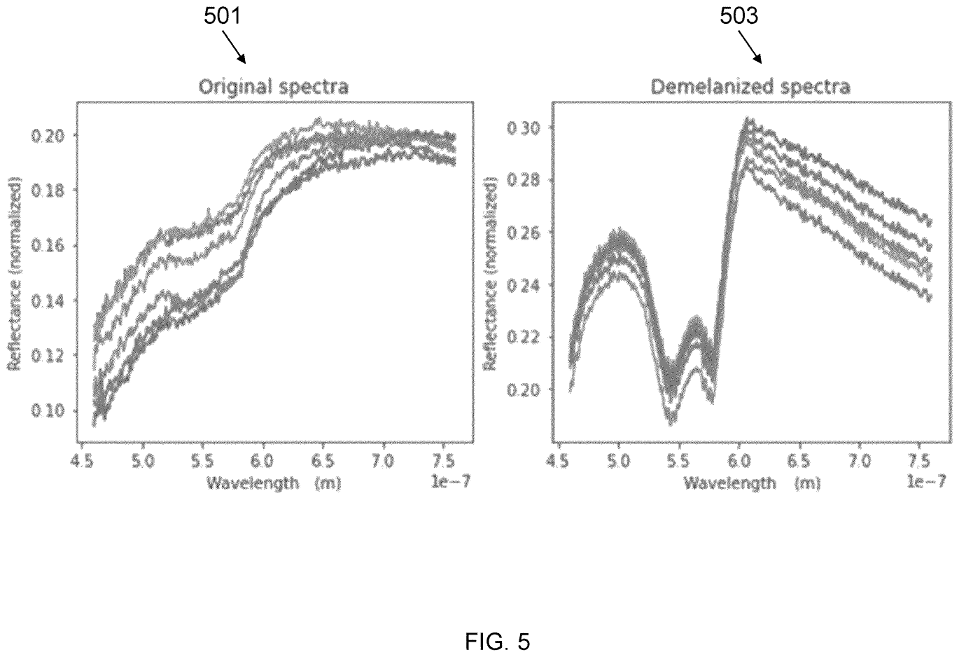

FIG. 5 shows plots of spectra before and after filtering using the trained machine learning model;

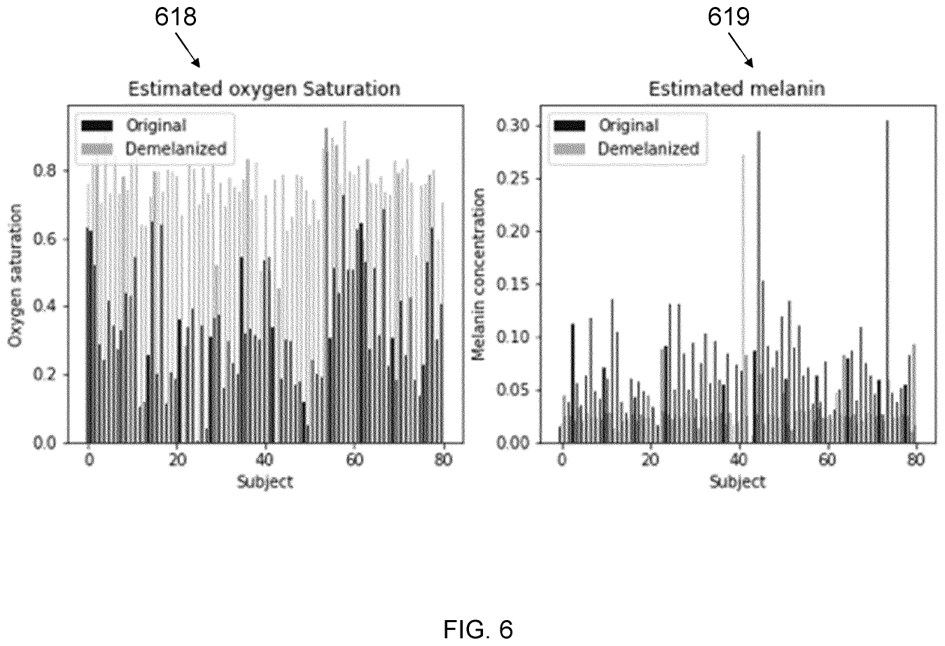

FIG. 6 shows two related graphs illustrating the effect of the trained machine learning model on oxygen saturation estimates and melanin estimates;

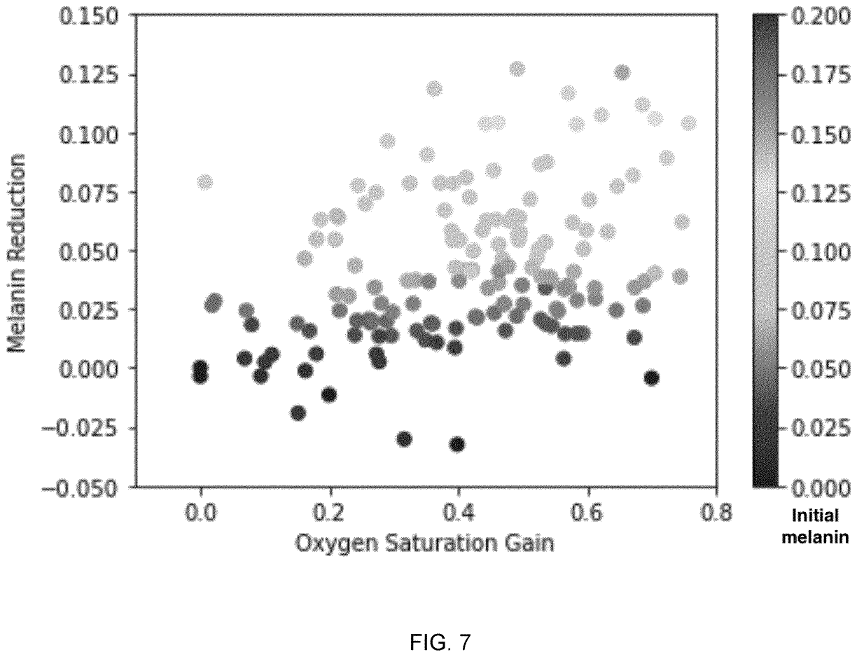

FIG. 7 is a plot showing the melanin reduction for different skin tones achieved using the trained machine learning model;

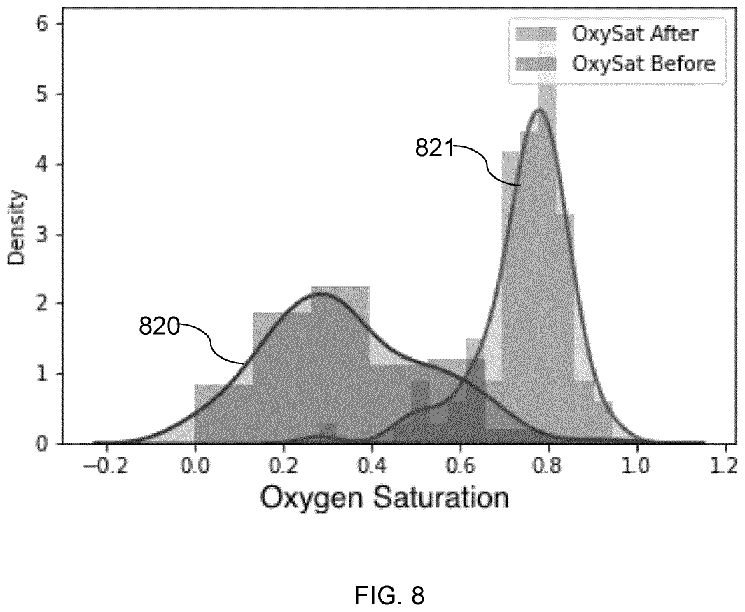

FIG. 8 is a graph showing SmVO2 distribution before and after filtering using the trained machine learning model; and

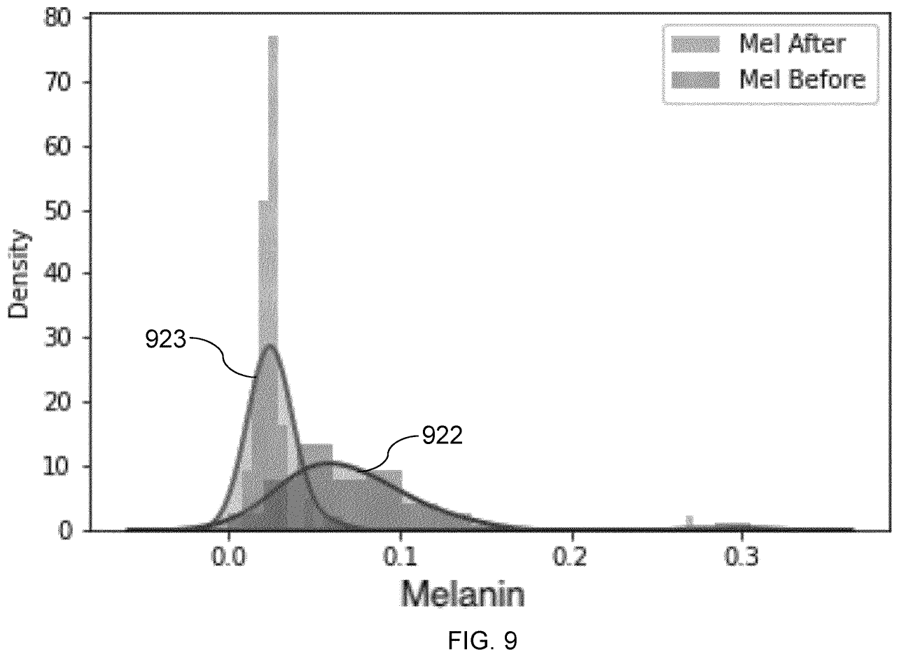

FIG. 9 is a graph showing melanin distribution before and after filtering using the trained machine learning model.

FIG. 1A is a schematic drawing illustrating how an oxygen saturation estimate can be obtained from a subject using an embodiment of the invention. FIG. 1A shows a human patient 150; a clinician 151; a Diffuse Reflectance Spectroscopy (DRS) probe 152; and a processing system 155 comprising a trained machine learning model 102, a DRS model 104 for estimating SmVO2 values, a readout device 154 and a memory 153.

The clinician 151 performs Diffuse Reflectance Spectroscopy by placing the DRS probe 152 on the patient's skin. The probe is connected to an off-the-shelf spectrometer (not shown), for example, the AvaSpec-Mini2048CL available from Avantes. The probe 152 transmits visible and NIR light from optical fibre(s) within the probe 152 and measures the reflectance and wavelength of the returned light to generate a reflectance spectrum. The patient 152, for example, has dark skin and a melanin concentration of 9% which would usually impact the accuracy of oxygen saturation readings when using known techniques.

The spectral data obtained by the probe 152 is input to the processing system 155 and put through the trained machine learning model 102 which filters the spectrum to reduce the effect of the melanin on the reflectance spectrum and to generate a filtered melanin-standardised spectrum. The filtered spectrum then gets put through a DRS model 104, operating in an inverse mode, which outputs an estimated SmVO₂ (oxygen saturation) value which can be presented to the clinician via the readout device 154 and/or saved to the memory 153, thereby allowing important clinical decisions to be taken.

A trained machine learning model according to an embodiment of the present invention is described herein and is referred to as ‘MEL-AE’ (i.e. MELanin AutoEncoder). The full-stack of the MEL-AE filtering technique is shown in the flow diagram shown in FIG. 1B.

FIG. 1B illustrates an example process of how spectra can be processed using a trained machine learning model and a deterministic software or mathematical model to give an oxygen saturation parameter estimate. In this example, the value of the melanin parameter is representative of the concentration of melanin in the skin of a human subject. The oxygen saturation parameter represents an SmVO₂ value.

FIG. 1B shows four Diffuse Reflectance Spectroscopy (DRS) spectra 101 from four different readings of the same subject and a ‘demelanised’ version 103 of those spectra. The spectra 101, 103 plot reflectance of light (from a body surface) on the y-axis and wavelength of said light on the x-axis.

Also shown in FIG. 1B is a MEL-AE machine learning model 102 and a DRS model 104 used in inverse mode. The DRS model 104 is a deterministic software or mathematical model which obtains an oxygen saturation parameter estimate from DRS spectra (in an inverse mode). The output of the process is a melanin-standardised SmVO₂ estimate 105.

The high-level operation of the process shown in FIG. 1B will now be described.

How the MEL-AE model 102 is trained to ‘demelanise’ the DRS spectra 101 will be described further down in more detail.

The MEL-AE trained machine learning model 102 receives (DRS) spectra 101 as input and generates melanin-standardised spectra 103 as output.

The melanin-standardised spectra 103 is then fed into an DRS model 104. The DRS model 104 generates a melanin-standardised SmVO₂ estimate which is a measure of oxygen saturation. The inverse approach in general calculates the optical properties of the skin based on the DRS spectrum. This is coupled with a mathematical model of transport of light through tissue. Using this information, estimates of oxygen saturation can be made.

The deterministic software or mathematical model 104 may also be operated in a ‘direct’ mode, where a set of parameters are transformed into a synthetic DRS spectrum. As will now be explained this is exploited in order to train the MEL-AE model 102.

FIG. 2 shows a diagram of the deterministic software or mathematical model 104. FIG. 2 also shows a set of input parameters 206 and a DRS spectrum 208 representative of reflectance (from a body surface) over a range of wavelengths.

The deterministic model 104 is so called because the set of parameters 206 that determine the spectra 208 are identifiable and may be reproduced by reversing the model in the aforementioned inverse mode.

Use of the DRS model 104 in direct mode is shown by the arrow going left to right. Use of the DRS model 104 in inverse mode is shown by the arrow going from right to left.

Having such a deterministic model 104, where the parameters that determine the spectra are known, it is possible at a low computational cost to operate the deterministic model 104 in a “generative” (direct) mode and produce spectra for a given set of parameters 206. The parameters may be representative of a scaling factor; calibrating light scattering effects and physiological properties such as the relative amount of blood (blood volume fraction, BVF), oxygen saturation and melanin.

The deterministic software or mathematical model 104 shown in FIG. 2 is suitable for training the machine learning model (the MEL-AE algorithm) 102 and for receiving the output of the trained machine learning model 102 during the process shown in FIG. 1B.

FIG. 3 illustrates how an embodiment of MEL-AE, in accordance with the invention is trained.

In FIG. 3 the MEL-AE training system 309 is shown to comprise a grid of admissible parameter values 310; a first set of parameters 311; a second set of parameters 312 and a DRS model 104 used in direct mode.

The grid of admissible values 310 includes allowed ranges of values (defined by ‘Min’ and ‘Max’ columns) for each parameter and an allowed resolution for each parameter. Training is executed off-line by generating two sets of input parameters 311, 312 from the grid 310 for the DRS model 104 to turn into a pair of training spectra. The two sets of input parameters which are used to generate the corresponding pair of training spectra only vary in that one has a predetermined melanin parameter (M1=3%) and the other can have a different melanin parameter. This process is repeated so that a plurality of pairs of training spectra may be obtained. The pairs of spectra always include one spectrum that is generated based on the ‘standard’ value of melanin concentration and a corresponding spectrum which could be based on any value of melanin concentration out of a range of values.

A first set of parameters 311 is used to generate a first training spectrum and a second set of parameters 312 is used to generate a second training spectrum. The first training spectrum represents an input spectrum for the trained machine learning model 102 with a given melanin concentration and the second training spectrum represents a desired melanin-fixed output spectrum. The aim of training is for the machine learning model to learn how to generate the desired melanin-fixed output spectrum from any DRS input spectrum.

The first set of parameters 311 contains N parameters which are selected (randomly or sequentially) from the grid 310 of admissible values. The first set of parameters are used to generate the reflectance spectra associated with that particular set of parameters 311 (the first training spectrum).

The second set of parameters 312 also contains N parameters. N-1 of the parameters are identical to the first set of parameters 311, but the melanin parameter (the Nth parameter) is set to a predetermined value (e.g. 3% is chosen here). This modified set of parameters 312 is used to generate a second training spectrum. The first and second training spectra act as the input 401 and target 403 for the autoencoder.

A training data-set made of a plurality of pairs of spectra, is therefore built (by random or exhaustive search) exploring the space of the N input parameters, based on the granularity associated for each parameter.

Each synthetic training spectrum is preferably chosen to be a square (of 1024 or 2048 pixels) in order to easily turn the spectra into a 2D matrix (32×32 or 64×64). This helps to speed up the training process.

Therefore, a deterministic DRS model 104 is used to generate a plurality of pairs of synthetic training spectra, each pair comprising a noisy spectrum (with melanin) and a denoised spectrum (with reduced melanin). These pairs can then train the autoencoder to provide melanin-fixed spectra.

The space where the clinical and optical parameters are allowed to vary is relatively small, given a reasonable precision required on the estimates. Therefore, it is not computationally expensive to prepare the training dataset or a validation dataset.

Furthermore, the training or validation dataset preparation only needs to be done once as the format of the input spectra is standardized.

A granularity of 50 levels per clinical parameter (e.g. 2% steps for oxygen saturation) will require only about 15 billion synthetic spectra to be generated.

Forcing the melanin concentration estimated from the actual data to be changed via a controlled ‘melanin parameter’ (e.g. having the melanin parameter reduced to a lower value), means it is possible to produce another spectrum based on a standard melanin concentration (e.g. a lower concentration of melanin).

FIG. 4 is a diagram of the trained machine learning model architecture and an example of input and output spectra for the model. FIG. 4 shows an input spectrum 401 with ‘unknown’ melanin; an output spectrum 403 with reduced melanin and an autoencoder comprised of an encoder 415 and a decoder 417.

FIG. 4 therefore shows, in an example embodiment, that the trained machine learning model 102 comprises two mirrored artificial neural network architectures 415,417 joined into an “autoencoder”. The encoder 415 reduces the dimensionality of the input reflectance spectrum 401 to a lesser dimension. This means the encoder 415 transforms the original spectrum 401 into a compressed version (i.e. in a reduced space 416) that includes the most relevant features. The decoder 417 then obtains the target output spectrum 403 from the compressed representation of the input spectrum 401.

If, for example, the target output spectra 208 are versions of the input spectra 403 with a predetermined lower melanin parameter, the autoencoder is trained to learn how to reduce the effect of melanin on input spectra accordingly. Thus, the trained machine learning model 102 is able to perform melanin adjustment on an input spectrum that it has not previously encountered.

FIGS. 5 to 9 show the results of experimental tests, performed on clinical data from human patients, of a melanin-filtering method embodying the invention.

The MEL-AE algorithm used for the testing was trained, as described above, off-line using synthetic spectral data generated by a DRS model in direct mode.

To generate a training set, a space of six model parameters (including melanin) was discretized (the granularity of each parameter depending on the physiological relevance of said parameter) and randomly sampled to extract a training dataset made up of Nₜᵣₐᵢₙ=6×10⁵ and Nₜₑₛₜ=2×10⁴ data points. Each point represents a combination of six parameters generating a pair of reflectance curves, the first having an estimated value of melanin (including M#3%) and the second having a standard value of melanin (M=3%).

Of the six parameters, three are associated with the DRS model. For instance, one parameter represents a scaling factor and two parameters are provided for calibrating the light scattering effects. Three parameters are representative of physiological properties (i.e. the relative amount of blood (blood volume fraction, BVF), oxygen saturation and melanin).

Using anonymised data coming from previously approved hospital-run studies, 80 subjects with differing skin tones (i.e. and thus different values of melanin concentration) were used for testing the algorithm. For each of these 80 subjects, 2 randomly selected spectra (out of 12 taken for each subject) were selected and oxygen saturation (SmVO₂) estimated (via the DRS model 104) using the method embodying the present invention.

FIG. 5 shows plots of spectra before and after filtering using the trained machine learning model. The first plot shows DRS spectra taken from real human patients.

The second plot 503 shows the first spectra after filtering through the MEL-AE model 102 to be representative of a standard value of melanin (M=3%).

The x-axes of the spectra 501, 503 in FIG. 5 represent wavelength of light in metres and the y-axis represents a normalised reflectance value (no units), i.e. the normalised ratio of the amount of light leaving the body surface to the amount of light striking the body surface.

The MEL-AE algorithm 102 was used to post-process the acquired spectra, and to generate filtered spectra which was used to estimate both SmVO₂ and melanin concentration. Since two spectra were selected for each subject, the numerical value calculated for SmVO₂ and melanin was averaged.

The double inverse peak (seen at a wavelength of approximately 540 nm- 576 nm) which is typical of spectral data oxygenated and de-oxygenated blood, is barely visible when inspecting dark skins. However, after filtering using MEL-AE this peak should again be visible (see 503 of FIG. 5). Therefore, by selecting several spectra coming from a subject with a dark skin tone, the effect of the MEL-AE filter on dark skin can be checked even before the parameter estimation.

Another effect of standardisation of the melanin is in FIG. 5. The post-processed spectra 503 are more similar compared to the recorded spectra 501. This may be a sign of a non-uniform spatial distribution of melanin locally in the skin. The reduction to a standard melanin-equivalent spectrum 503 for each of them, helps to remove also minor fluctuations, helping stabilize the SmVO₂ estimates.

The output signal (see 503) may contain some noise, as a side effect of the dimensionality reduction, which can be easily cleaned by the use of standard filters (e.g. Savitzky-Golay) before calculating the parameters for the demelanised version of the spectrum. This signal processing noise has been found by the applicant to have a limited effect on the accuracy of the calculations of the parameters.

Additionally, the dimensionality reduction associated with the usage of an autoencoder helps to mitigate local artifacts in the input spectra inducing lower fluctuations in the clinical parameters calculated for the output spectra.

FIG. 6 is show two related graphs illustrating the effect of the trained machine learning model on SmVO₂ estimates 618 and melanin estimates 619.

The x-axis of each of the histograms 618, 619 represents the individual test subjects (80 in total). The y-axis of the first plot 618 represents oxygen saturation expressed as a fraction. The y-axis of the second plot 619 represents melanin concentration expressed as a fraction. On each histogram 618, 619 original data and demelanised data are shown (see legend).

As can be seen in the composite histogram of estimated oxygen saturation 618 in FIG. 6, the effect of the trained machine learning model (MEL-AE) is also apparent in the change of estimated concentration of SmVO₂ when comparing the original histogram to the demelanised histogram. The ‘demelanisation’ (performed by the trained machine learning model) generally provides an increase of the estimates of oxygen saturation. This is in line with the concept of ‘demelanisation’ reducing the coverage effect (e.g. screening or shielding of haemoglobin) due to melanin.

In the composite histogram of estimated melanin 619 it can be seen that no matter the skin tone/melanin levels recorded, the melanin for the filtered spectra tends to oscillate around the target 3% prescribed in the training. This demonstrates successful training of the MEL-AE algorithm.

FIG. 7 is a plot showing the melanin reduction for different skin tones achieved using the trained machine learning model.

The applicant has identified that the efficiency of the melanin filtering performed by the trained machine learning model is proven to be dependent on the melanin levels themselves. When dealing with highly pigmented skins the effect of melanin removal is more pronounced and generally higher SmVO₂ are reported (see FIG. 7).

FIG. 8 is a graph showing SmVO₂ distribution before and after filtering using the trained machine learning model. FIG. 9 is a graph showing melanin distribution before and after filtering using the trained machine learning model.

The overall effect of melanin reduction, SmVO₂ change and standardisation of their value associated with MEL-AE over the dataset can be understood by looking at the histograms in FIG. 8 and FIG. 9.

The raw recorded oxygen saturation and melanin distributions 820, 922 show a broader spread than their corresponding distributions 821, 923 obtained after being put through the MEL-AE model 102.

As the dataset included both healthy and ill subjects, the “normal” values for the SmVO₂ are not necessarily around a single “normal” value but may have a long or fat tail due the presence of ongoing medical conditions (see FIG. 8). In contrast, the distribution 923 of melanin, after using the trained machine learning model, shown in FIG. 9 is indeed forced to concentrate around the 3% by design.

It will be appreciated by those skilled in the art that the invention has been illustrated by describing one or more specific embodiments thereof, but is not limited to these embodiments; many variations and modifications are possible, within the scope of the accompanying claims.

1. A computer-implemented method of processing spectral data to determine an estimated value of an oxygen saturation parameter, the method comprising:
i) receiving a spectrum obtained by a spectroscopic method performed on a body surface of a subject;
ii) inputting said spectrum to a trained machine learning model, the model having been trained by a method comprising:
generating or receiving synthetic training data comprising a plurality of pairs of spectra, each pair comprising:
a first training spectrum determined by a first set of parameters, wherein the first set of parameters are deterministic of the first training spectrum, the first set of parameters comprising a melanin parameter;
a second training spectrum determined by a second set of parameters, wherein the second set of parameters are deterministic of the second training spectrum; wherein the second set of parameters are the first set of parameters with the melanin parameter set to a predetermined value; and
using the training data to train the machine learning model to generate a modified version of said spectrum corresponding to said melanin parameter having said predetermined value; and
iii) generating said estimate of the oxygen saturation parameter using said trained machine learning model.

2. The computer-implemented method of claim 1, comprising the step of performing the spectroscopic method on the subject.

3. The computer-implemented method of claim 1 or 2, wherein the input spectrum is a reflectance spectrum obtained by diffuse reflectance spectroscopy performed on a body surface of the subject.

4. The computer-implemented method of any preceding claim, wherein the trained machine learning model is trained to generate a filtered spectrum, wherein the melanin parameter is set to the predetermined value.

5. The computer-implemented method of any preceding claim, comprising generating the estimate of the oxygen saturation parameter by a deterministic software or mathematical model operating in an inverse mode, the deterministic software or mathematical model being arranged to operate in the inverse mode or in a direct mode.

6. The computer-implemented method of claim 5, comprising the deterministic software or mathematical model generating, in the direct mode, the first training spectrum from the first set of parameters and the second training spectrum from the second set of parameters.

7. The computer-implemented method of any preceding claim, comprising using the trained machine learning model in conjunction with a or the deterministic software or mathematical model to obtain an estimation of oxygen saturation.

8. The computer-implemented method of any preceding claim, wherein the melanin parameter represents a measure of a melanin concentration in the body surface of the subject.

9. The computer-implemented method of any preceding claim, wherein the predetermined value of the melanin parameter is between 1% and 5%.

10. The computer-implemented method of any preceding claim, wherein the predetermined value of the melanin parameter is one of the following:
a) a value for which a double peak in a reflectance spectrum is most easily discernible; or
b) a value which allows an oxygen saturation parameter to be obtained that is substantially standardised for all skin tones.

11. The computer-implemented method of any preceding claim, wherein the trained machine learning model comprises an unsupervised neural network which is an autoencoder.

12. The computer-implemented method of any preceding claim, wherein the method of training the machine learning model further comprises selecting each of the first set of parameters and the second set of parameters from a space of admissible values.

13. The computer-implemented method of claim 12, wherein the space of admissible values comprises, for each parameter of the set of parameters, a permitted range of values for each parameter of the set of parameters, wherein
each permitted range is represented in the space by a minimum value and a maximum value for each parameter; and/or
the space of admissible values further comprises a permitted resolution for each parameter.

14. The computer-implemented method of any of claims 12 or 13, wherein the first set of parameters contains N parameters which are selected randomly or sequentially from the admissible values, and the second set of parameters also contains N parameters, wherein N-1 of the parameters are identical to the first set of parameters but the melanin parameters (the Nth parameter) is set to the predetermined value.

15. The computer implemented method of any of claims 12 to 14, wherein each set of parameters comprise parameters representative of one or more of the group comprising:
a) a scaling factor;
b) calibrating light scattering effects and physiological properties such as the relative amount of blood (blood volume fraction);
c) oxygen saturation; and
d) melanin.

16. The computer-implemented method of any of claims 14 or 15, wherein the parameters from each set of parameters have a granularity of between 10 and 100 levels, and the training data is prepared by a random or exhaustive search exploring the space of permitted values of the N parameters, based on the granularity associated with each parameter.

17. The computer-implemented method of any preceding claim, wherein at least some of the training and input spectra are input as square images.

18. A non-transitory computer-readable storage medium comprising instructions that, when executed by a processor, cause the processor to carry out the method of any preceding claim.

19. A processing system comprising one or more processors and a memory storing software for execution by the one or more processors, wherein the processing system is configured to perform the method of any of claims 1 to 17.

20. A computer-implemented method of training a machine learning model, the method comprising:
generating or receiving synthetic training data comprising a plurality of pairs of spectra, each pair comprising:
a first training spectrum determined by a first set of parameters, wherein the first set of parameters are deterministic of the first training spectrum, the first set of parameters comprising a melanin parameter; and
a second training spectrum determined by a second set of parameters, wherein the second set of parameters are deterministic of the second training spectrum; wherein the second set of parameters are the first set of parameters with the melanin parameter set to a predetermined value; and
using the training data to train the machine learning model to generate:
an estimate of a value of an oxygen saturation parameter from a spectrum obtained by a spectroscopic method performed on a body surface of a subject; or
data suitable for determining said estimate.

21. A computer-implemented trained machine learning model, the model having been trained by a method comprising:
generating or receiving synthetic training data comprising a plurality of pairs of spectra, each pair comprising:
a first training spectrum determined by a first set of parameters, wherein the first set of parameters are deterministic of the first training spectrum, the first set of parameters comprising a melanin parameter; and
a second training spectrum determined by a second set of parameters, wherein the second set of parameters are deterministic of the second training spectrum; wherein the second set of parameters are the first set of parameters with the melanin parameter set to a predetermined value; and
using the training data to train the model to generate:
an estimate of a value of an oxygen saturation parameter from a spectrum obtained by a spectroscopic method performed on a body surface of a subject; or
data suitable for determining said estimate.
